(19) United States
(12) Patent Application Publication
(10) Pub. No.: US 2016/0045684 A1
(43) Pub. Date: Feb. 18, 2016

(54) Inhaler For Powdered Medicaments

(71) Applicant: NiPPharma Co., Ltd., Tokyo (JP)

(72) Inventor: Shinichi ONO, Tokyo (JP)

(21) Appl. No.: 14/462,546

(22) Filed: Aug. 18, 2014

(51) Int. Cl. A61M 15/00

(52) U.S. Cl. CPC A61M 15/0021; A61M 15/0045; A61M 2202/064; A61M 2202/0057; A61M 2209/084

(57) ABSTRACT

One object of the present invention is to provide an inhaler which is able to stand by itself with a simple composition. The present invention is directed to an inhaler (1) for powdered medicaments. The inhaler (1) comprises a housing (2) enclosing the medicaments and a holder (3) movably mounted to the housing (2). The housing (2) includes a mouthpiece (20) having an inlet aperture (21) for the medicaments. The holder (3) is movable between an open configuration in which the mouthpiece (20) is exposed and a closed configuration in which the mouthpiece (20) is covered with the holder (3). The holder (3) includes a stand portion (30) to make the housing (2) stand on a flat surface.

### TECHNICAL FIELD

The present invention relates to an inhaler for powdered medicaments. The inhaler of the present invention has a mouthpiece and a holder. The holder is movable so as either to cover the mouthpiece to protect it when the inhaler is stored, or to expose the mouthpiece for use.

### BACKGROUND OF THE INVENTION

An inhaler which is appropriate for delivering medicaments in dry powder form used in the treatment of respiratory diseases, particularly in asthma and chronic obstructive pulmonary disease (COPD) is already known. It is also well known to use the inhaler for delivering medicaments utilized in the treatment and prophylaxis of respiratory diseases by the oral route. Inhalation treatment is the most commonly preferred treatment method in these diseases because the inhaler is easy to use. The powdered medicaments have more rapid onset of time resulting from local administration and they also have fewer side effects.

For example, a conventional inhaler is disclosed in Patent document 1. The conventional inhaler comprises a housing including a mouthpiece and a cover movably mounted to the housing. The cover is movable between an open position in which the mouthpiece is exposed for use and a closed position in which the mouthpiece is enclosed. In the open position, the cover at least partly defines an inlet aperture to an inhalation air flow path leading through the inhaler and communicating with the mouthpiece. In the closed position, the cover covers the mouthpiece to protect it when the inhaler is stored in a shelf or a bag, for example.

### CITATION LIST

### Patent Literature

Patent document 1: International Publication No. WO 2011-129787

### SUMMARY OF INVENTION

### Technical Problem

However, the cover of the conventional inhaler has an overall disc-like shape. Due to the disc-like shape of the cover, the inhaler is liable to fall down and it is unable to stand by itself. Therefore, it is impossible to store the inhaler to a shelf or a bag without falling down. If the inhaler lies down in the shelf or the bag, there is a possibility that the powdered medicaments which remains in the inhaler leak out from the housing and the cover.

Therefore, one object of the present invention is to provide an inhaler which is able to stand by itself with a simple composition. Another object of the present invention is to inhibit leaking out of the powdered medicaments.

### Solution to Problem

The present invention is directed to an inhaler (1) for powdered medicaments. The inhaler (1) comprises a housing (2) enclosing the medicaments and a holder (3) movably mounted to the housing (2). The housing (2) includes a mouthpiece (20) having an inlet aperture (21) for the medicaments. The holder (3) is movable between an open configuration in which the mouthpiece (20) is exposed and a closed configuration in which the mouthpiece (20) is covered with the holder (3). The holder (3) includes a stand portion (30) to make the housing (2) stand on a flat surface.

It is preferable that the stand portion (30) is wider than all other portions of the housing (2), and the stand portion (30) has a flat plane in a bottom region.

It is preferable that the stand portion (30) includes a weight member (32). It is also preferable that the stand portion (30) includes an adsorption member (33) to which the medicaments are absorbed, instead of the weight member (32) or together with the weight member (32).

### Advantageous Effects of Invention

The present invention can provide an inhaler which is able to stand by itself with a simple composition. The inhaler of the present invention can inhibit leaking out of the powdered medicaments from the housing or the holder.

### BRIEF DESCRIPTION OF DRAWINGS

FIG. 1(a) and FIG. 1(b) are top perspective views of the inhaler of the present invention. FIG. 1(a) shows the inhaler in open configuration. FIG. 1(b) shows the inhaler in closed configuration.

FIG. 2(a) and FIG. 2(b) are front views of the inhaler of the present invention. FIG. 2(a) shows the inhaler in open configuration. FIG. 2(b) shows the inhaler in closed configuration.

FIG. 3 shows a weight member and/or an adsorption member in a preferred embodiment of the present invention.

### DESCRIPTION OF EMBODIMENTS

One Embodiment for carrying out the present invention is explained with the figures as follows. However the present invention is not restricted within the following embodiments, and involves proper arrangements of the following embodiments by a skilled person.

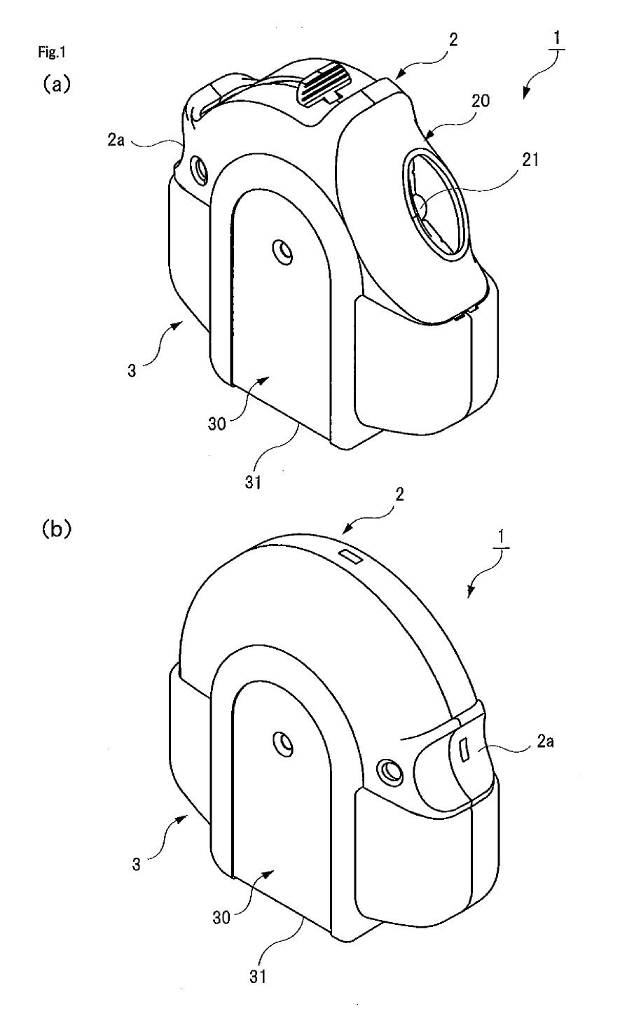

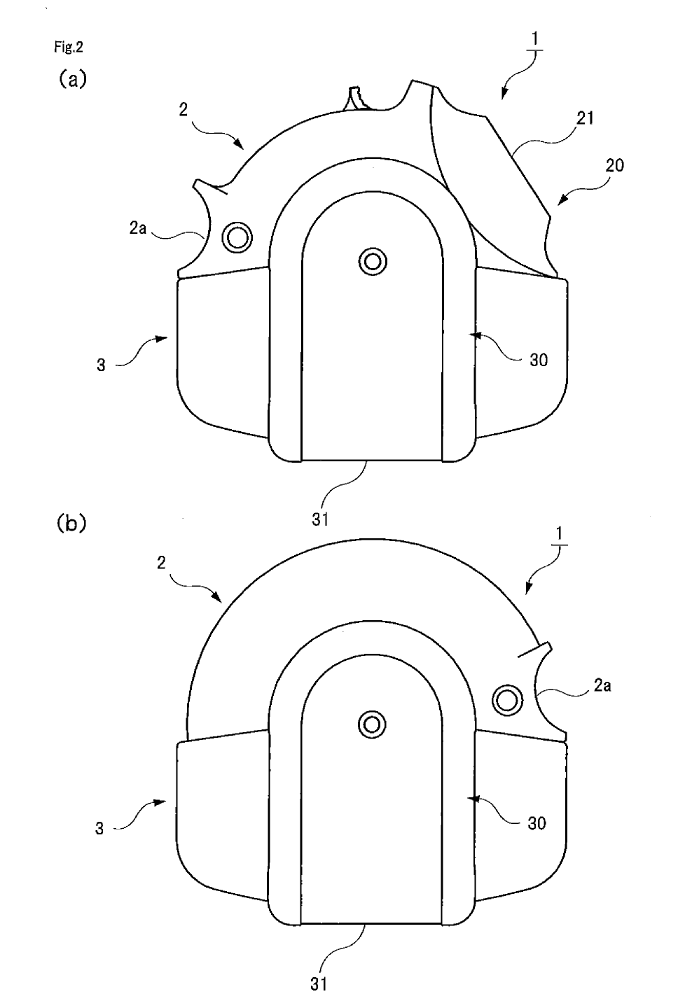

FIG. 1 and FIG. 2 show one embodiment of an inhaler (1) of the present invention. The inhaler (1) is appropriate for delivering medicaments in dry powder form used in the treatment of respiratory diseases. Examples of the respiratory diseases are asthma and chronic obstructive pulmonary disease (COPD).

FIG. 1(a) and FIG. 2(b) show the inhaler (1) of the present invention in open configuration. FIG. 1(b) and FIG. 2(b) show the inhaler (1) of the present invention in closed configuration. As shown in FIG. 1 and FIG. 2, the inhaler (1) is configured from a housing (2) and a holder (3). The housing (2) encloses the medicaments therein. The holder (3) is movably mounted to the housing (2). The housing (2) includes a mouthpiece (20) having an inlet aperture (21) for the medicaments. The holder (30) is movable between the open position in which the mouthpiece (20) is exposed and the closed position in which the mouthpiece (20) is covered with the holder (30).

The inhaler (1) enables delivery of the medicaments in dry powder form from blister package (not shown in the figures), for example. Thus the blister package is enclosed in the housing (2). The blister package has already been well known. The blister package is provided to carry the medicaments in dry powder form. The blister package is composed of blister pockets each of which generally contain one dose of medicaments in dry powder form. The dry powdered medicament in the blister pocket that is opened upon the actuation of the inhaler (1) is generally entrained to the mouthpiece (20) through the manifold.

The housing (2) further includes a gear mechanism (not shown in the figures). The gear mechanism has some gears to enable the inhalation of the dry powder medicaments carried in the blister package. The gear mechanism has already been well known. For example, the mechanism comprises indexing wheel and a ratchet wheel. The indexing wheel which engages with the ratchet wheel enables the blister package to be indexed. The blister pockets composing the blister package are received in the recesses on the indexing wheel. And then, the blister package is indexed when the indexing wheel rotates. The blister package is peeled by the indexing wheel and one blister pocket is opened in response to each actuation of the gear mechanism.

The holder (3) is relatively movable to the housing (2). As shown in FIG. 1 and FIG. 2, the holder (3) is movably mounted to the housing (2). In other words, the housing (2) (together with the mouthpiece (20)) are arranged to pivot with respect to the holder (3) between the closed position in which the mouthpiece (10) is covered with the holder (3) and the open position in which the mouthpiece (10) is exposed for use. The inhaler (1) shown in FIG. 1 (a) and FIG. 2 (a) is ready for the inhalation. In this case, the holder (3) is in the open position and the mouthpiece (10) is entirely exposed. The housing (2) has to be rotated by holding on the carved part (2a) on one end of the housing (2) in order to switch to the open position from the closed position wherein the mouthpiece (20) is completely covered with the holder (3). In this way, the mouthpiece (20) is completely exposed when it is switched to the open position from the closed position. Then, the gear mechanism in the housing (2) is driven.

As shown in FIG. 1 and FIG. 2, the holder (3) includes a stand portion (30). The stand portion (30) makes the inhaler (1) possible to stand on a flat surface by itself. The stand portion (30) is constructed by a part of the shape of the holder (3). The stand portion (30) is positioned in the central region of the holder (3). In this embodiment, the stand portion (30) is wider than the other parts of the holder. For example, the depth of the stand portion (30) is preferably from 110% to 200% of the depth of the other parts of the holder.

The stand portion (30) which is wider than the other parts is at least partly having a flat plane (31). The flat plane (31) is positioned at least in the bottom region of the stand portion (30). Therefore the flat plane (31) of the stand portion (30) faces to a flat surface on which the inhaler (1) is placed. The inhaler (1) having the flat plane (31) at the bottom of the holder (3) is able to stand erect on the flat surface without any other supports. The inhaler (1) is easy to store in a shelf or bag, for example, because it is resistant to fall down in the shelf etc. The present invention can provide an inhaler (1) which is able to stand by itself with a simple composition.

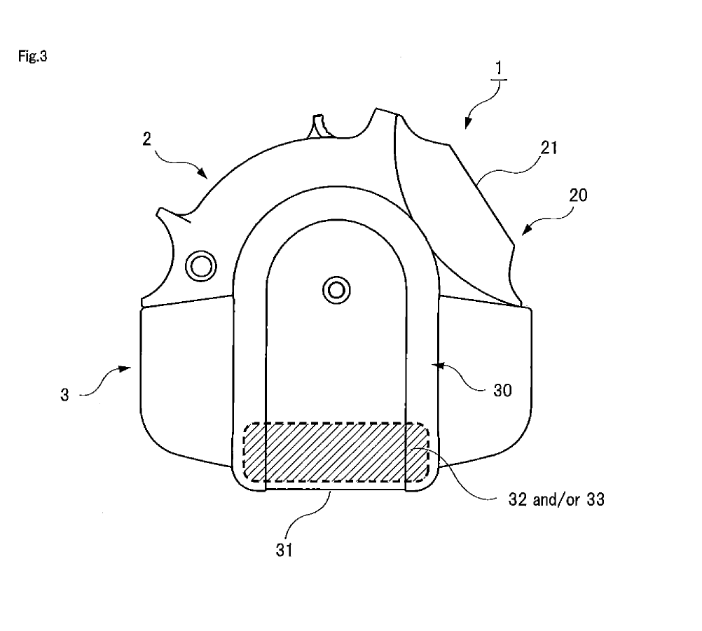

FIG. 3 shows a preferred embodiment of the present invention. In the preferred embodiment, the inhaler (1) further comprises a weight member (32) within the stand portion (30) of the holder (3). As shown in FIG. 3, the weight member (31) is arranged on the inner side of the flat plane (31) of the stand portion (30). It is preferred that the weight member (32) consists of metal such as iron, copper, and stainless steel. The weight member (32) may be made of magnet material. As a result of arranging the weight member (32) within the stand portion (30), the stability of the inhaler (1) which stands on the flat surface is improved. Furthermore, by placing the weight member (31) made of the magnet material therein, since the inhaler (1) is able to be stuck to metal plate with the magnet, the usability of the inhaler (1) is more improved.

It is possible to replace the weight member (32) with an adsorption member (33) to which the medicaments are absorbed. Since the absorption member (33) can absorb the powdered medicaments scattered within the inhaler (1), the inhaler (1) can inhibit leaking out of the powdered medicaments from the housing (2). Examples of the adsorption member (33) are adhesive compound, adhesive tape, and electrostatically-charged plastic sheet. The absorption member (33) may be arranged within the stand portion (30) together with the weight member (32).

### INDUSTRIAL APPLICABILITY

The present inhaler may be used in the pharmaceutical industry.

1 Inhaler

2 Housing

3 Holder

20 Mouthpiece

21 Inlet aperture

30 Stand portion

31 Flat plane

32 Weight member

33 Absorption member

1. An inhaler for powdered medicaments comprising:
a housing enclosing the medicaments; and
a holder movably mounted to the housing,
wherein the housing includes a mouthpiece having an inlet aperture for the medicaments,
wherein the holder is movable between an open configuration in which the mouthpiece is exposed and a closed configuration in which the mouthpiece is covered with the holder, and
wherein the holder includes a stand portion to make the housing stand on a flat surface.

2. The inhaler in accordance with claim 1, wherein the stand portion is wider than all other portions of the housing, and the stand portion has a flat plane in a bottom region.

3. The inhaler in accordance with claim 1, wherein the stand portion includes a weight member.

4. The inhaler in accordance with claim 1, wherein the stand portion includes an adsorption member to which the medicaments are absorbed.
